(19) United States
(12) Patent Application Publication
(10) Pub. No.: US 2020/0138875 A1
(43) Pub. Date: May 7, 2020

(54) AGENT FOR TREATING MICROBIOME-RELATED DISEASES

(71) Applicants: Horst Przuntek, Bochum (DE); Aiden Haghikia, Bochum (DE)

(72) Inventors: Horst Przuntek, Bochum (DE); Aiden Haghikia, Bochum (DE)

(21) Appl. No.: 16/612,517

(22) PCT Filed: May 25, 2018
(86) PCT No.: PCT/EP2018/063817

§ 371 Date: Nov. 11, 2019

(30) Foreign Application Priority Data
May 26, 2017 (DE) 10 2017 111 547.1

(51) Int. Cl. A61K 35/74; A61K 9/00

(52) U.S. Cl. CPC A61K 35/74; A61K 9/0053

(57) ABSTRACT

The invention relates to an agent for use in the treatment of diseases due to an imbalance of the microbiome, in particular multiple sclerosis (MS) and Parkinson's disease, in the form of a microbiome transplant to be administered to the patient to be treated, wherein the microbiome transplant being obtainable from the stool of one or several healthy subjects through extraction with an aqueous medium, concentration of the extract and packaging to obtain a transplantable concentrate.

The invention relates to an agent for use in the treatment of diseases due to an imbalance of the microbiome, in particular multiple sclerosis and Parkinson's disease.

Numerous illnesses are associated with a deficient colonization of the intestine, that is the microbiome of the intestine is out of balance, whether due to a deficient colonization or the loss of essential constituents for the maintenance of bodily functions. In the case of all these diseases, such as obesity, ulcerative colitis, multiple sclerosis (MS), Parkinson's disease and presumably also psychoses, but also in rheumatic disease forms and psoriasis, significant changes in the microbiome of the intestine can be detected. This suggests that microbiome and the respective disease are interlinked, either as a result of a common cause or due to reciprocally acting influencing factors.

The invention is based on the findings that in both multiple sclerosis and Parkinson's disease a change in the microbiome has occurred compared to healthy control subjects. Braak has postulated that Parkinson's disease begins in the intestine. This is in line with the observation that the transfer of the microbiome from mice with Parkinson's disease to healthy mice produces a comparable picture of Parkinson's disease.

Alpha-synuclein, a transport protein that occurs in the brain and plays a role in Parkinson's disease, can be detected in very early stages of the disease in the intestine and serves as an indicator. This also suggests a correlation.

This analogously applies to multiple sclerosis. In this case, the microbiome can even be used and serves to differentiate between exacerbation patients and chronically progressive patients. In this case as well, the clinical picture of multiple sclerosis can be produced in animal experiments through the application of an MS microbiome to healthy animals.

In the event of patients suffering from ulcerative colitis the colitis could be eliminated by microbial transplantation. However, an acute colitis not only represents a more threatening clinical picture than multiple sclerosis and Parkinson's disease, it is also a direct disease of the intestine. It was therefore not the obvious or advisable approach to apply this procedure directly to patients suffering from neurological diseases, such as multiple sclerosis and Parkinson's disease, especially since the opinion prevailing in professional circles consider changes in the intestinal flora and neurological diseases to have a common cause.

Experiments have been conducted to compensate for deficits in intestinal colonization by taking medicinal or dietary measures, but this has only been successful to a limited extent.

It was found that through transplantation of a healthy intestinal flora to patients suffering from multiple sclerosis and Parkinson's disease a positive effect on the course of the disease could be achieved.

When transplanting the microbiome of a healthy person to a patient, the risks must be carefully weighed not only with respect to the relevant clinical pictures. In this context it must be ruled out that together with the microbiome pathogenic microorganisms are transferred, which also includes other disease risks such as those arising from the genetic disposition of the donor for example. Aside from this, it must be ensured that the transplanted microbiome comprises the entire spectrum of the microflora and that there are no deficiencies in important aspects or that it is thinned out/diluted.

On the basis of examinations, the quality of the microbiome can be determined to a limited extent only. Considering the fact that there are more than 20,000 species of microorganisms in a healthy microbiome, it is impossible to carry out relevant examinations with respect to completeness. In this respect, only an examination targeting on key organisms may provide more conclusive information.

It is therefore the objective of the present invention to provide a microbial transplant that on the one hand is capable of effectively treating the disease of the recipient, in particular Parkinson's disease and multiple sclerosis, and, on the other, of minimizing the patient's risk of being exposed to other diseases or defects that may be transmitted.

This objective is achieved with an agent of the kind first mentioned above, in which the microbial transplant is obtained from the stool of one or several healthy subjects by extraction with an aqueous medium, concentration of the extract and packaging to obtain a transplantable concentrate.

The risk of transmitting a microbiome-induced disease can be reduced by using the microbiome of many “healthy” subjects to produce the transplant. This principle of risk abatement is known from the production of immunoglobulins. Therefore, it is as a rule of significance to make use of the microbiome of a plurality of subjects, especially more than 50, preferably 50 to 100.

It is, moreover, essential that the subjects had not been treated with antibiotics for a period of at least three months, preferably longer, as these have an influence on the composition of the microbiome. Also of significance is that the subjects are healthy individuals in the sense that they do not have any diseases that could presumably be associated with an altered intestinal flora. This applies in particular to gastrointestinal complaints and disorders as well as typical intestinal diseases.

It must additionally be ensured that the microbiome of the subjects does not contain any pathogenic microorganisms, as, for example, those found with typical intestinal diseases that are caused by salmonella and campylobacter and the like. It is also useful and expedient to perform a routine genetic screening of the subjects.

The agent proposed by the invention is particularly successful in the treatment of multiple sclerosis and Parkinson's disease.

With regard to producing/manufacturing the agent it is expedient to proceed in several steps. At first, the stool is removed from the subject using a sterile collection tray and, after taking a test sample, deep-frozen within 20 minutes at −80° C.

Provided a stool sample is approved and released, suitable stools are placed in a sieve and extracted using a rinsing solution. Water is particularly suitable as a rinsing solution. The residue is discarded, with the liquid being collected and frozen again at −80° C. until further use. It may be useful to freeze the individual solutions in portions, for example in quantities of 100 ml.

In the event the extracts have to be transported, transportation must take place in an uninterrupted cold chain.

For transplantation purposes, extracts are preferably concentrated, for example by gentle differential centrifugation or freeze-drying.

The microbiome containing extracts may be administered directly rectally, however, they are preferably concentrated before packaging, if necessary to the dry state. The concentrates may also be administered rectally in liquid form or orally in encapsulated form. When administered orally, care must be taken to ensure that the capsules are not attacked by gastric acid, but that they enter the intestines with their contents unchanged and only dissolve therein.

With respect to the treatment of patients, multiple transplantations aimed at “normalizing” the intestinal flora are usually required. During this period, it is of prime importance to treat the patient with antibiotically acting substances only when it is absolutely necessary.

When handling and storing the extracts, it is important that temperatures are maintained at −80° C. and that the packaged concentrate is unfreezed only directly before treatment or examinations. It is absolutely necessary to make sure the cold chain is maintained.

As far as the subjects are concerned, there are only a few health-related restrictions. Preference is given to subjects who consume a healthy and varied diet, especially without meat.

1. Agent for use in the treatment of diseases due to an imbalance of the microbiome, in particular multiple sclerosis (MS) and Parkinson's disease, in the form of a microbiome transplant to be administered to the patient to be treated, wherein the microbiome transplant being obtainable from the stool of one or several healthy subjects through extraction with an aqueous medium, concentration of the extract and packaging into a transplantable concentrate.

2. Agent according to claim 1, obtainable from the stool of several subjects.

3. Agent according to claim 1, obtainable from the stool of more than 50 subjects.

4. Agent according to claim 1, characterized in that the disease is multiple sclerosis or Parkinson's disease.

5. Agent according to claim 1, obtainable from the stool of subjects free from intestinal diseases.

6. Agent according to claim 1, obtainable from the stool of subjects who have not undergone antibiotic therapy for a period of at least three months.

7. Agent according to claim 1, characterized in that an analysis of the microbiome is conducted before the extraction takes place.

8. Agent according to claim 1 that is obtainable by centrifugation and/or freeze-drying of the extract.

9. Agent according to claim 1 in liquid form for rectal infusion.

10. Agent according to claim 1 in solid form, encapsulated for oral administration.
